(19) United States
(12) Patent Application Publication
(10) Pub. No.: US 2013/0325046 A1
(43) Pub. Date: Dec. 5, 2013

(54) MEDICAL DEVICE

(71) Applicant: Cook Medical Technologies LLC, Bloomington, IN (US)

(72) Inventors: Matthew J. Terwiske, Bloomington, IN (US); Lynn K. Nolte, Bloomington, IN (US); Melissa Dale, Linton, IN (US); Jorge Jimenez, Bloomington, IN (US); Daniel K. Whetham, Bloomington, IN (US); Charles W. Agnew, West Lafayette, IN (US)

(21) Appl. No.: 13/908,330

(22) Filed: Jun. 3, 2013

Related U.S. Application Data
(60) Provisional application No. 61/655,766, filed on Jun. 5, 2012.

(51) Int. Cl. A61B 17/32

(52) U.S. Cl. CPC A61B 17/32
USPC 606/167

(57) ABSTRACT

A medical device may include a sheet member having a first surface and a second surface to engage a surface of a body tissue. The medical device may include an incision guide extending along a length of the sheet member. First and second portions of the sheet member may be positioned on respective first and second sides of the incision guide. The sheet member may be severable along the incision guide to separate the first portion and the second portion from one another. A first magnetic member may be positioned on the first portion, and a second magnetic member may be positioned on the second portion. The first and second magnetic members may be attracted to one another to magnetically couple the first portion and the second portion to one another. The medical device may include an alignment mechanism for aligning the separated first and second portions with one another.

### CROSS-REFERENCE TO RELATED APPLICATIONS

This application claims priority and the benefit of provisional U.S. Patent Application Ser. No. 61/655,766, filed Jun. 5, 2012, which is incorporated herein by reference in its entirety.

### TECHNICAL FIELD

This disclosure relates generally to medical devices. More specifically, this disclosure relates to surgical devices for guiding an incision through a body tissue and aiding in alignment of the tissue planes for closure of the incision.

### BACKGROUND

A variety of medical procedures include making an incision through a body tissue. To close the incision, the physician generally aligns the tissue planes on opposite sides of the incision and holds the tissue planes together with forceps. While holding the tissue planes together, the physician may suture the planes to one another to close the incision.

One medical procedure which includes making an incision is a cesarean section, or C-section. Such a procedure is an increasingly common alternative to natural childbirth (i.e., vaginal delivery). During a cesarean section, an incision is made through a patient's abdomen and uterus to deliver a child. After delivery of the child, the physician must close the incision through the uterine tissue (i.e., the hysterotomy). As is common when closing incisions through other body tissues, the physician generally will use forceps to hold the planes of uterine tissue on either side of the incision together while using a needle driver to pass a needle through the tissue to suture the incision closed.

Proper approximation of the uterine tissue (i.e., alignment of the tissue planes) following a cesarean section is important to promote proper healing of the uterine defect. If proper healing is not achieved, complications may occur in later pregnancies or later in life. Such complications may include abnormal placental plastentation, pelvic pain, or other complications.

### SUMMARY

The present embodiments provide a surgical device for guiding an incision through a body tissue and aiding in alignment of the tissue planes for closure of the incision.

In one example, a medical device may include a sheet member. The sheet member may include a first surface and a second surface positioned opposite the first surface to engage a surface of a body tissue. The medical device may include an incision guide extending along a length of the sheet member. A first portion of the sheet member may be positioned on a first side of the incision guide, and a second portion of the sheet member may be positioned on a second side of the incision guide opposite the first side. The sheet member may be severable along the incision guide to separate the first portion of the sheet member and the second portion of the sheet member from one another. The medical device may include a first magnetic member positioned on the first portion of the sheet member and a second magnetic member positioned on the second portion of the sheet member. The first magnetic member and the second magnetic member may be attracted to one another to magnetically couple the first portion of the sheet member and the second portion of the sheet member to one another. The medical device may include an alignment mechanism for aligning the first portion of the sheet member with the second portion of the sheet member after separation of the first portion and the second portion from one another.

In another example, a medical device may include a sheet member. The sheet member may include a first surface and a second surface positioned opposite the first surface to engage a surface of a body tissue. The medical device may include an incision guide extending along a length of the sheet member. A first portion of the sheet member may be positioned on a first side of the incision guide. A second portion of the sheet member may be positioned on a second side of the incision guide opposite the first side. The incision guide may include an opening through the sheet member extending along a portion of a length of the incision guide. The first portion of the sheet member may include a first edge extending along the opening. The second portion of the sheet member may include a second edge extending along the opening and positioned opposite the first edge. The first edge may include a first projection. The second edge may include a second projection. The sheet member may be severable along the incision guide to separate the first portion and the second portion of the sheet member from one another. The first projection and the second projection may be positionable relative to one another to realign the first portion and the second portion of the sheet member with one another after separation of the first portion and the second portion from one another.

In another example, a method for performing a surgical procedure may include applying a medical device to a surface of a body tissue. The medical device may include a sheet member. The sheet member may include an engaging surface engaged with the surface of the body tissue. The method may include cutting along an incision guide of the medical device to make an incision in a portion of the body tissue aligned with the incision guide. A first plane of the body tissue may be positioned on a first side of the incision. A second plane of the body tissue may be positioned on a second side of the incision opposite the first side. The method may include separating a first portion of the sheet member positioned on a first side of the incision guide and engaged with the first plane of the body tissue from a second portion of the sheet member positioned on a second side of the incision guide opposite the first side and engaged with the second plane of the body tissue to separate the first plane of the body tissue and the second plane of the body tissue from one another. The method may include magnetically coupling the first portion of the sheet member and the second portion of the sheet member to one another to align the first plane of the body tissue and the second plane of the body tissue with one another.

Other systems, methods, features, and advantages of the invention will be, or will become, apparent to one with skill in the art upon examination of the following figures and detailed description. It is intended that all such additional systems, methods, features, and advantages be within the scope of the invention, and be encompassed by the following claims.

### BRIEF DESCRIPTIONS OF THE DRAWINGS

FIG. 1 illustrates one example of a medical device having a plurality of protrusions arranged in one exemplary configuration.

FIG. 2 illustrates another example of a medical device having a plurality of protrusions arranged in another exemplary configuration.

FIG. 3 illustrates another example of a medical device having a plurality of protrusions arranged in another exemplary configuration.

FIG. 4 illustrates another example of a medical device having a plurality of protrusions arranged in another exemplary configuration.

FIG. 5 illustrates a cross sectional view of one example of a medical device having one exemplary incision guide and positioned on a body tissue.

FIG. 6 illustrates a cross sectional view of another example of a medical device having another exemplary incision guide and positioned on a body tissue.

FIG. 7 illustrates the medical device of FIG. 1 positioned on a body tissue.

FIG. 8 illustrates a portion of a medical device affixed to a body tissue with one example of a nail.

FIG. 9 illustrates a portion of a medical device including a barb in a predeployment configuration.

FIG. 10 illustrates the portion of the medical device of FIG. 9 with the barb in a deployed configuration.

FIG. 11 illustrates closure of an incision using the medical device of FIG. 1 as a suture guide.

FIG. 12 illustrates another example of a medical device.

### DETAILED DESCRIPTION OF THE DRAWINGS AND THE PRESENTLY PREFERRED EMBODIMENTS

Detailed embodiments of the present invention are disclosed herein. It is to be understood, however, that the disclosed embodiments are merely exemplary of the invention, which may be embodied in various and alternative forms. The figures are not necessarily to scale, and some figures may be configured to show the details of a particular component. Therefore, specific structural and functional details disclosed herein are not to be interpreted as limiting, but merely as a representative basis for the claims and for teaching one skilled in the art to practice the present invention.

In the present disclosure, the term “proximal” refers to a direction that is generally toward a physician during a medical procedure, while the term “distal” refers to a direction that is generally toward a target site within a patient's anatomy during a medical procedure.

FIG. 1 illustrates one example of a medical device 100, which may be used to aid in approximating planes of body tissue for closure of an incision through the body tissue. The medical device 100 may include a sheet member 110. The sheet member 110 may be a relatively thin sheet of material and may include a first surface 112 and a second surface 114 positioned opposite the first surface. The second surface 114 may be configured to engage a surface of the body tissue as further described below. For example, the sheet member 110 may be configured as a strip or bio-decal which may be attached to the uterus prior to making an incision therein as further described below. The sheet member 110 may have sufficient flexibility to enable the sheet member to be rolled, folded, bent, warped, or otherwise deformed. In one example, the sheet member 110 may be sufficiently flexible to conform to the engaged surface of the body tissue.

The sheet member 110 may have any size and/or shape. For example, the sheet member 110 may be substantially rectangular as shown in FIG. 1. In other words, the sheet member 110 may include four edges, with adjacent edges positioned at approximately right angles relative to one another. The four edges of the sheet member 110 may include a first end edge 116, a second end edge 117 positioned opposite the first end edge, a first side edge 118, and a second side edge 119 positioned opposite the first side edge. The end edges 116, 117 may extend along a width of the sheet member 110, and the side edges 118, 119 may extend along a length of the sheet member. In other examples, the sheet member 110 may have a circular, elliptical, triangular, or any other polygonal or non-polygonal shape. The corners of the sheet member 110 may be rounded as shown in FIG. 1. In other words, one or more of the corners of the sheet member 110 may be curved along a radius of curvature. This may reduce the probability of damaging the body tissue in contact with the sheet member 110.

The sheet member 110 may have a length A and a width B as shown in FIG. 1. The length A may be about the same length as a normal uterine incision. In one example, the length A may be between about 5 cm and about 18 cm, preferably between about 10 cm and about 12 cm. Additionally, or alternatively, the width B may be between about 1 cm and about 10 cm, typically between about 3 cm and about 7 cm, preferably about 5 cm. In other examples, the sheet member 110 may have any other suitable length and/or width. The sheet member 110 may have a thickness C as shown in FIG. 5. In one example, the thickness C may be less than about 1 mm (about 0.04 in), preferably less than about 0.5 mm (about 0.02 in). In other examples, the sheet member 110 may have any other suitable thickness. The thickness of the sheet member 110 may be sufficiently large to encapsulate one or more magnets as further described below.

The medical device 100 may include an incision guide 120, which may extend along the length of the sheet member 110. In one example, the incision guide 120 may extend along substantially the entire length of the sheet member 110 as shown in FIG. 1. In other examples, the incision guide may extend along any portion of the length of the sheet member 110. The incision guide 120 may aid a physician in making an incision through a body tissue to which the medical device 100 may be attached. For example, the incision guide may serve as a template for making an incision in a body tissue as further described below. In one example, the incision guide 120 may include an opening 122 and an indicator 124 as shown in FIG. 1. A portion of the length of the incision guide 120 may be defined by the opening 122, and another portion of the length of the incision guide may be defined by the indicator 124. In other words, the opening 122 and the indicator 124 may cooperatively define the incision guide 120.

The opening 122 may be configured as an aperture disposed in the sheet member 110. The opening 122 may extend through the thickness of the sheet member from the first surface 112 to the second surface 114. The opening 122 may extend along a portion of the length of the sheet member 110 to define a portion of the length of the incision guide 120. The opening 122 may have any suitable shape and/or size. For example, the opening may have a rectangular, circular, elliptical, triangular, or any other polygonal or non-polygonal shape. The opening may have a length D and a width E. In one example, the length D may be between about 8 cm and about 15 cm, preferably between about 10 cm and about 13 cm. Additionally, or alternatively, the width E may be between about 3 cm and about 6 cm, preferably between about 3.5 cm and about 5.5 cm. In other examples, the opening 122 may have any other suitable length and/or width.

The indicator 124 may be configured as a mark disposed on the sheet member 110 (e.g., on the first surface 112 and/or the second surface 114). The mark may be configured as a cutting mark as further described below. The indicator 124 may extend along a portion of the length of the sheet member 110 to define a portion of the length of the incision guide 120. In one example, the indicator 124 may include two indicators as shown in FIG. 1. One indicator 124 may be positioned at one end of the sheet member 110 (e.g., near the first end edge 116), and the other indicator 124 may be positioned at an opposite end of the sheet member (e.g., near the second end edge 117). The opening 122 may be positioned between the two indicators 124. Each indicator 124 may extend from an edge of the sheet member 110 (e.g., the respective end edge of the sheet member) to the opening 122. In this manner, the incision guide 120, which may include the opening 122 and the two indicators 124, may extend along substantially the entire length of the sheet member 110. In other words, the incision guide 120 may extend from the first end edge 116 to the second end edge 117 of the sheet member 110. The indicator 124 may be configured to indicate to a physician a location where the sheet member 110 should be cut to make an incision as further described below. In one example, the indicator 124 may be configured as a mark disposed on the first surface 112 of the sheet member 110 as shown in FIG. 1. In another example, the indicator 124 may be configured as a groove or an indentation disposed in the sheet member as further described below.

The incision guide 120 may divide the sheet member 110 into a first portion 130 positioned on a first side of the incision guide and a second portion 140 positioned on a second side of the incision guide opposite the first side. In other words, the incision guide 120 may define a border or boundary between the first portion 130 and the second portion 140 of the sheet member 110. The first portion 130 and the second portion 140 may be separated from one another by severing the sheet member 110 along the incision guide 120 as further described below. In one example, the incision guide 120 may be positioned approximately in the center of the sheet member 110 with respect to the width thereof so that each of the first portion 130 and the second portion 140 of the sheet member forms approximately one-half of the sheet member as shown in FIG. 1. In other examples, the incision guide 120 may be positioned at any other location with respect to the width of the sheet member 110 such that the first portion 130 and the second portion 140 may form any fraction of the sheet member. In one example, the first portion 130 and the second portion 140 of the sheet member 110 may be mirror images of one another as shown in FIG. 1. In other words, the sheet member 110 may be symmetrical with respect to the incision guide 120. In other examples, the sheet member 110 may be asymmetrical with respect to the incision guide 120.

The first portion 130 of the sheet member 110 may include an edge 132 extending along the opening 122 of the incision guide 120. A perimeter of the first portion 130 may include the edge 132, a portion of the end edge 116 of the sheet member 110, the side edge 118 of the sheet member, and a portion of the end edge 117 of the sheet member as shown in FIG. 1. The edge 132 may define a portion of a perimeter of the opening 122. In other words, the edge 132 may define the boundary between the opening 122 and the first portion 130 of the sheet member 110. The edge 132 may include a side portion 133 extending along the length of the opening 122, a first end portion 134 extending along a portion of the width of the opening, and a second end portion 135 positioned opposite the first end portion and extending along a portion of the width of the opening.

The second portion 140 of the sheet member 110 may include an edge 142 extending along the opening 122 of the incision guide 120. A perimeter of the second portion 140 may include the edge 142, a portion of the end edge 116 of the sheet member 110, the side edge 119 of the sheet member, and a portion of the end edge 117 of the sheet member as shown in FIG. 1. In other words, the edge 142 may define the boundary between the opening 122 and the second portion 140 of the sheet member 110. The edge 142 may define a portion of the perimeter of the opening 122 positioned opposite the portion of the perimeter defined by the edge 132. The edges 132, 142 may collectively define the perimeter of the opening 122. The edge 142 may include a side portion 143 extending along the length of the opening 122, a first end portion 144 extending along a portion of the width of the opening, and a second end portion 145 positioned opposite the first end portion and extending along a portion of the width of the opening.

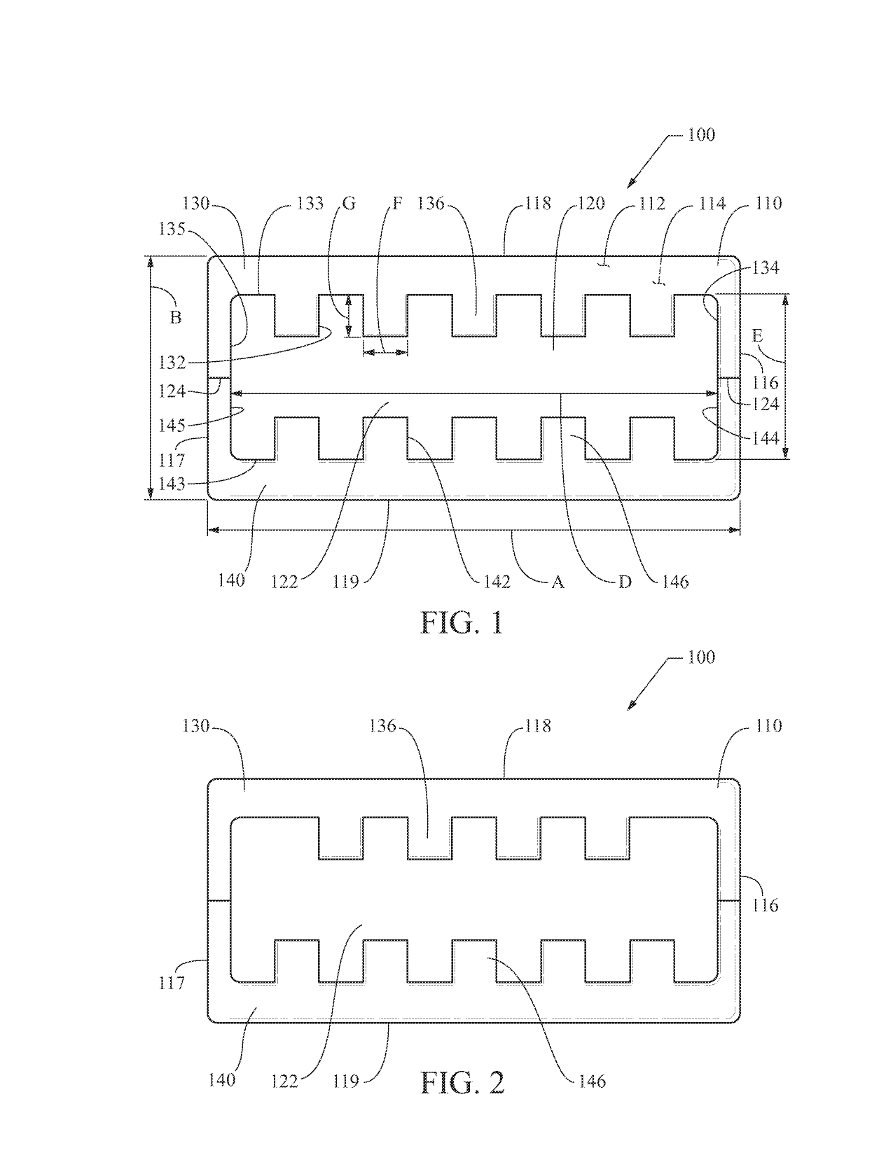

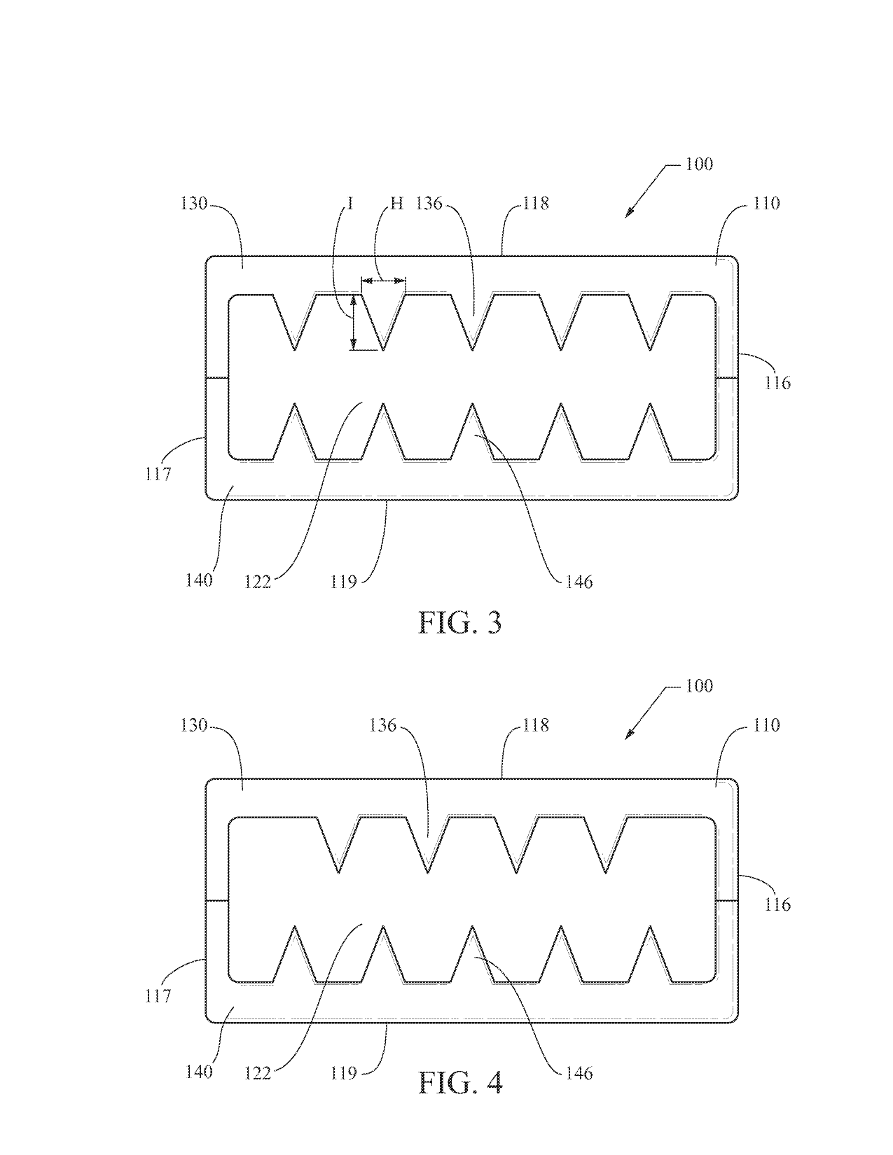

In one example, the first portion 130 of the sheet member 110 may include one or more projections 136. FIGS. 1-4 illustrate various exemplary configurations of the projections 136. Each projection 136 may be configured as an extension or protrusion of the first portion 130 into the opening 122. The projection 136 may extend into the opening 122 such that the width of the portion of the opening into which the projection extends may be less than the width E of the opening. The side portion 133 of the edge 132 may have a nonlinear configuration to define the projections 136. In other words, the side portion 133 of the edge 132 may be nonlinear such that the width of the opening 122 varies along the length of the opening. The projections 136 may have any suitable shape and/or size. In one example, each projection 136 may have a substantially rectangular shape as shown in FIGS. 1-2. To that end, each projection 136 may include a pair of side edges joined to one another by an end edge. Each side edge of the projection 136 may be substantially perpendicular to the end edge of the projection. The end edge of the projection 136 may be substantially parallel to the side edge 118 of the sheet member 110. In another example, each projection 136 may have a substantially triangular shape as shown in FIGS. 3-4. To that end, each projection 136 may include a pair of edges extending away from the side edge 118 of the sheet member 110 into the opening 122 and intersecting one another to form an acute angle therebetween. In other examples, the projections may have any other suitable shape.

In one example, each projection 136 may have a length F and a width G as shown in FIG. 1. In one example, the length F may be between about 2 mm and about 12 mm, preferably between about 3 mm and about 10 mm. Additionally, or alternatively, the width G may be between about 4 mm and about 15 mm, preferably between about 5 mm and about 13 mm. In another example, each projection 136 may have a length H and a width I as shown in FIG. 3. The length H may correspond to a base of the substantially triangular projection 136. Additionally, or alternatively, the width I may correspond to a height of the substantially triangular projection 136. In one example, the length H may be between about 2 mm and about 12 mm, preferably between about 3 mm and about 10 mm. Additionally, or alternatively, the width I may be between about 4 mm and about 15 mm, preferably between about 5 mm and about 13 mm. In other examples, the projection 136 may have any other suitable length and/or width.

The projections 136 may be spaced from one another along the length of the opening 122. For example, the projections 136 may be spaced from one another along the length of the side portion 133 of the edge 132 as shown in FIGS. 1-4. The spacing between adjacent projections 136 may be given as a distance (e.g., along the side portion 133 of the edge 132) between adjacent projections. In other words, the spacing between adjacent projections 136 may be given as the distance by which one projection is spaced from an adjacent projection. In one example, such a distance may be the distance between adjacent side edges of adjacent substantially rectangular projections. In another example, such a distance may be the distance between the bases of adjacent substantially triangular projections. In one example, the projections 136 may be spaced from one another by a distance of between about 2 mm and about 12 mm, preferably between about 5 mm and about 8 mm. In other examples, the projections 136 may be spaced from one another by any other suitable distance. Alternatively, the spacing between adjacent projections 136 may be given as a distance on center. The distance on center between adjacent projections 136 may be the distance between the approximate center (e.g., approximately the center of the end edge of the substantially rectangular projection or the intersection between the pair of edges of the substantially triangular projection) of one projection and the approximate center of an adjacent projection.

The projections 136 may be arranged such that the first portion 130 of the sheet member 110 has a comb-like shape as shown in FIGS. 1-4, with the projections 136 resembling the teeth of the comb. In this manner, the projections 136 may form a first guide comb of the first portion 130 of the sheet member 110 extending along the opening 122 of the incision guide 120. The first guide comb may include one or more recesses which may be defined by portions of the opening 122 positioned between adjacent projections 136. The projections 136 and the recesses positioned therebetween may collectively define the first guide comb. The first guide comb may function as a suture guide to aid a physician in closing an incision as further described below.

Additionally, or alternatively, the second portion 140 of the sheet member 110 may include one or more projections 146 as shown in FIGS. 1-4. Each projection 146 may be configured as an extension or protrusion of the second portion 140 into the opening 122. The projection 146 may extend into the opening 122 such that the width of the portion of the opening into which the projection extends may be less than the width E of the opening. The side portion 143 of the edge 142 may have a nonlinear configuration to define the projections 146. In other words, the side portion 143 of the edge 142 may be nonlinear such that the width of the opening 122 varies along the length of the opening. The projections 146 may have any suitable shape and/or size as described above with reference to the projections 136. The projections 146 may be spaced from one another along the length of the opening 122 as described above with reference to the projections 136. For example, the projections 146 may be spaced from one another along the length of the side portion 143 of the edge 142 as shown in FIGS. 1-4.

The projections 146 may be arranged such that the second portion 140 of the sheet member 110 has a comb-like shape as shown in FIGS. 1-4, with the projections 146 resembling the teeth of the comb. In this manner, the projections 146 may form a second guide comb of the second portion 140 of the sheet member 110 extending along the opening 122 of the incision guide 120. The second guide comb may include one or more recesses which may be defined by a portion of the opening 122 positioned between adjacent projections 146. The projections 146 and the recesses positioned therebetween may collectively define the second guide comb. The second guide comb may function as a suture guide to aid a physician in closing an incision as further described below.

In one example, the first portion 130 of the sheet member 110 may include the projections 136, and the second portion 140 of the sheet member may include the projections 146 as shown in FIGS. 1-4. The side portions 133, 143 of the edges 132, 142 of the respective first portion 130 and second portion 140 of the sheet member 110 may be positioned opposite one another with respect to the opening 122 such that the projections 136, 146 extend inward into the opening and toward one another. In other words, the projections 136 may extend into the opening 122 in a first direction away from the side edge 118 of the sheet member 110, and the projections 146 may extend into the opening in a second direction opposite the first direction and away from the side edge 119 of the sheet member. In this manner, the first guide comb of the first portion 130 of the sheet member 110 and the second guide comb of the second portion 140 of the sheet member may face one another as shown in FIGS. 1-4. The first guide comb and the second guide comb may collectively form a suture guide which may aid a physician in closing an incision as further described below.

In one example, one projection 136 of the first portion 130 may be substantially aligned with a corresponding projection 146 of the second portion 140 as shown in FIGS. 1 and 3. For example, the center of the end edge of one projection 136 of the first portion 130 may be substantially aligned with the center of the end edge of a corresponding projection 146 of the second portion 140 with respect to the length of the sheet member 110 as shown in FIG. 1. Also for example, the intersection of the pair of edges of one projection 136 of the first portion 130 may be substantially aligned with the intersection of the pair of edges of a corresponding projection 146 of the second portion 140 with respect to the length of the sheet member 110 as shown in FIG. 3. The corresponding projections 136, 146 may be spaced from one another with respect to the width of the sheet member 110. In other words, the corresponding projections may be spaced such that the ends of the corresponding projections extending into the opening 122 are spaced from one another as shown in FIGS. 1-4. In one example, the first guide comb of the first portion 130 and the second guide comb of the second portion 140 may have the same number of teeth and may be aligned with one another such that each projection 136 of the first portion 130 is substantially aligned with a corresponding projection 146 of the second portion 140 as shown in FIGS. 1 and 3.

In another example, one projection 136 of the first portion 130 may be offset from a corresponding projection 146 of the second portion 140 as shown in FIGS. 2 and 4. For example, a side edge of one projection 136 of the first portion 130 may be substantially aligned with an opposite side edge of a corresponding projection 146 of the second portion 140 as shown in FIG. 2. In other words, the end edge of one projection 136 of the first portion 130 may be substantially aligned with a recess positioned between two adjacent projections 146 of the second portion 140. Also for example, the intersection of the pair of edges of one projection 136 of the first portion 130 may be substantially aligned with the recess positioned between two adjacent projections 146 of the second portion 140 as shown in FIG. 4. In one example, the first guide comb and the second guide comb may be offset from one another such that each tooth of the first guide comb is misaligned with a corresponding tooth of the second guide comb as shown in FIGS. 2 and 4.

In one example, the first portion 130 of the sheet member 110 may include one or more end recesses as shown in FIGS. 1-4. For example, a first end recess may be defined by a portion of the opening 122 positioned between a projection 136 positioned adjacent to the first end portion 134 of the edge 133 and the first end portion 134. Additionally, or alternatively, a second end recess may be defined by a portion of the opening 122 positioned between a projection 136 positioned adjacent to the second end portion 135 of the edge 133 and the second end portion 135. Similarly, the second portion 140 of the sheet member 110 may include one or more end recesses. For example, a first end recess may be defined between a projection 146 positioned near the first end portion 144 of the edge 143 and the first end portion 144. Additionally, or alternatively, a second end recess may be defined between a projection 146 near the second end portion 145 of the edge 143 and the second end portion 145. The end recesses of the first portion 130 and the second portion 140 of the sheet member 110 may be at least partially aligned with one another to aid a physician in beginning and/or ending a suture as further described below.

In other examples, the first portion 130 and the second portion 140 of the sheet member 110 may have any number of projections 136, 146 (e.g., the same number or a different number), and the projections may be arranged in any manner relative to one another (e.g., aligned, offset, or any position therebetween). Any number of recesses may be defined between the projections 136 of the first portion 130 and/or the projections 146 of the second portion 140.

FIGS. 5-11 illustrate the medical device 100 shown in FIG. 1 in use to aid a physician in making and/or closing an incision in a body tissue during a surgical procedure. In one example, the surgical procedure may be a cesarean section. An incision may be made through the patient's abdomen to expose the uterine wall 200. The medical device 100 may be placed on the uterine wall 200. For example, the medical device 100 may be placed on an outer surface of the uterine wall such that the second surface 114 of the sheet member 110 engages the outer surface of the uterine wall as shown in FIGS. 5-7. In one example, the second surface 114 of the sheet member 110 may engage the uterine wall 200 such that the sheet member is affixed to the uterine wall. In other words, the sheet member 110 may be attached to the uterine wall 200 by the engagement between the second surface 114 and the uterine wall. The sheet member 110 may be affixed to the uterine wall 200 in any suitable manner. In one example, the second surface 114 of the sheet member 110 may include an adhesive which may affix the second surface of the sheet member to the outer surface of the uterine wall 200 upon placement of the sheet member on the uterine wall. The adhesive may include any suitable medical adhesive known in the art such as, for example, a hypoallergenic pressure sensitive adhesive commercially available from 3M™, St. Paul, Minn. In one example, the medical adhesive may be graded for less than 24 hours.

Additionally, or alternatively, the sheet member 110 may be affixed to the uterine wall 200 by a mechanical attachment mechanism. In one example, the mechanical attachment mechanism may include a nail or tack 150 as shown in FIG. 8. The nail 150 may include a head portion 152 and a sharpened tip portion 154 extending from the head portion. The tip portion 154 of the nail 150 may be pushed through the sheet member 110 of the medical device 100 such that the tip portion penetrates the outer surface of the uterine wall 200 and the sheet member 110 is captured between the head portion 152 and the uterine wall. In this manner, the sheet member 110 may be affixed to the uterine wall 200. The nail 150 may be formed from any suitable material such as, for example, a polymeric material.

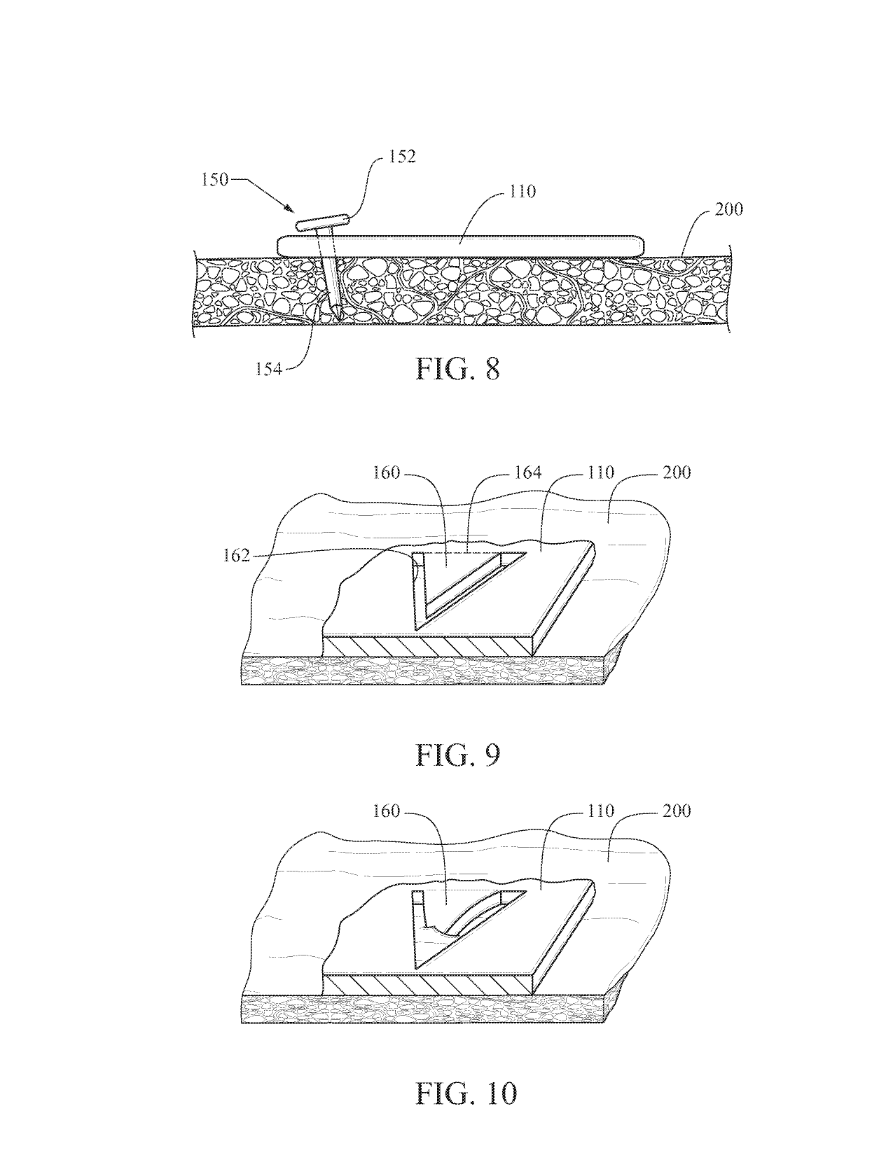

In another example, the mechanical attachment mechanism may include one or more barbs 160 as shown in FIGS. 9-10. The barb 160 may be formed integrally with or separately from the sheet member 110. In one example, the barb may be formed integrally with the sheet member 110 by cutting a substantially V-shaped notch 162 in the sheet member as shown in FIG. 9. The barb 160 may have a substantially triangular shape with the base of the triangle joined to the sheet member 110 and the sides of the triangle positioned within the V-shaped notch 162. The base of the barb 160 may be configured as a bending line 164. The barb 160 may be movable (e.g., bendable) along the bending line 164 between a predeployment configuration and a deployed configuration. In the predeployment configuration, the barb 160 may be substantially coplanar with the sheet member 110 as shown in FIG. 9. In the deployed configuration, the barb 160 may be bent relative to the sheet member 110 along the bending line 164 such that the barb 160 is non-coplanar with the sheet member. For example, the barb 160 may extend away from the plane of the sheet member and toward the uterine wall 200 as shown in FIG. 10. The tip of the barb 160 may be sharpened such that the barb may penetrate the outer surface of the uterine wall 200 when the sheet member 110 is placed on the uterine wall to affix the sheet member to the uterine wall. Multiple mechanical attachment mechanisms may be distributed about the sheet member 110 (e.g., at the corners and/or along the edges) to affix the sheet member to the uterine wall 200.

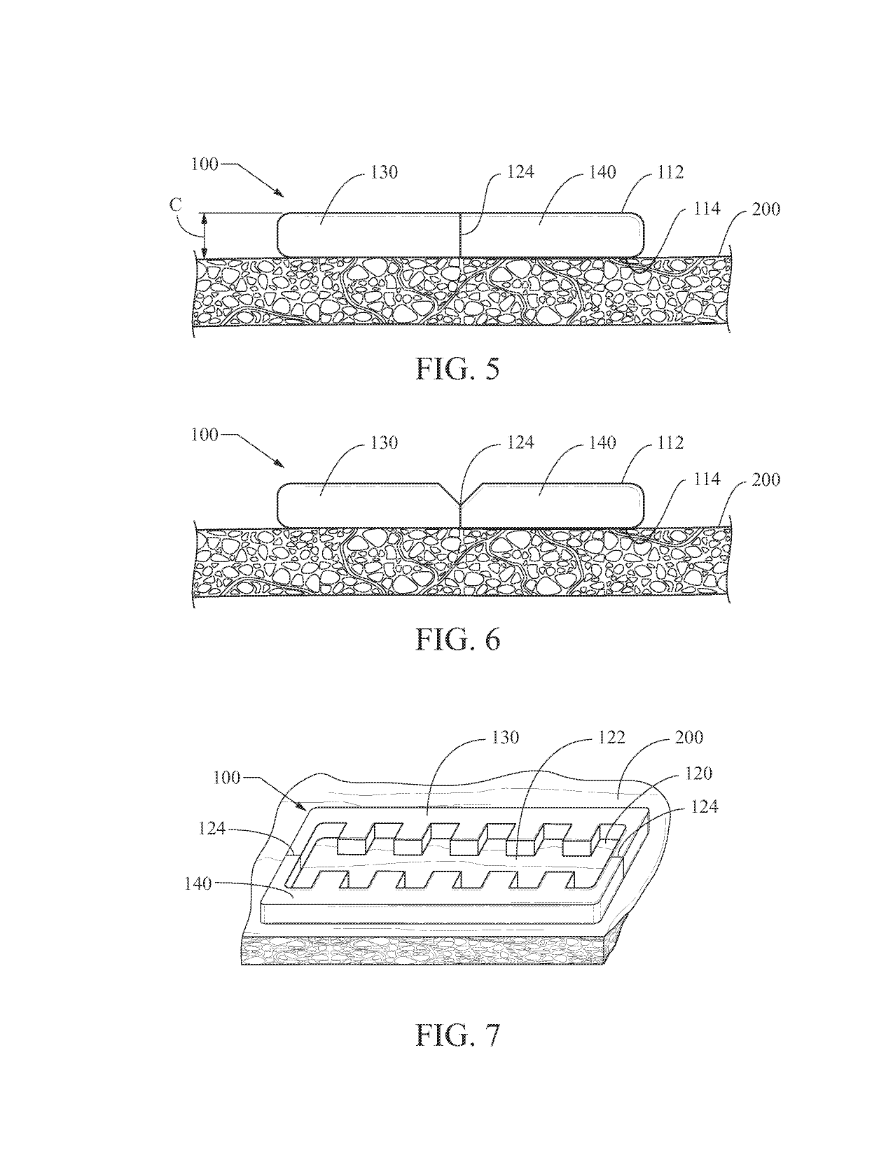

The sheet member 110 may be placed on the uterine wall 200 directly over the location where an incision is to be made. With the sheet member 110 in place, the physician may make the incision through the uterine wall 200. The incision guide 120 may guide the physician in making the incision. In other words, the incision guide 120 may function as a template for making the incision. For example, the physician may begin the incision by cutting (e.g., with a scalpel) through the sheet member 110 and the portion of the uterine wall 200 aligned with the sheet member along the indicator 124 of the incision guide 120. In one example, the indicator 124 may include a mark disposed on the sheet member as shown in FIGS. 5 and 7 and described above. The physician may begin the incision by cutting into the sheet member 110 at the mark disposed thereon. Additionally, or alternatively, the indicator 124 may include a portion of the sheet member 110 extending along at least a portion of the length of the sheet member and having a reduced thickness relative to the remaining portion of the sheet member. For example, the indicator 124 may include a groove or a channel as shown in FIG. 6. The physician may begin the incision by cutting into the groove in the sheet member 110. The reduced thickness of the sheet member 110 along the indicator 124 may reduce the resistance to severing sheet member which may aid the physician in making the incision.

The physician may continue the incision by severing the portion of the uterine wall 200 aligned with the opening 122 of the incision guide 120. In other words, the physician may cut in a generally lengthwise direction along the opening 122 to continue the incision along the portion of the uterine wall aligned with the opening. In one example, the physician may continue the incision by passing the scalpel between the first and second guide combs. The physician may further continue the incision by severing the sheet member 110 and the uterine wall 200 along the other indicator 124 as described above. In other words, the physician may cut the sheet member 110 and the portion of the uterine wall aligned with the sheet member along the incision guide 120 from the first end edge 116 of the sheet member and along the incision guide 120 to the second end edge 117 of the sheet member. In this manner, the first portion 130 and the second portion 140 of the sheet member 110 may be separated from one another. In other words, the physician may separate the first portion 130 and the second portion 140 from one another by severing the sheet member 110 along the incision guide 120.

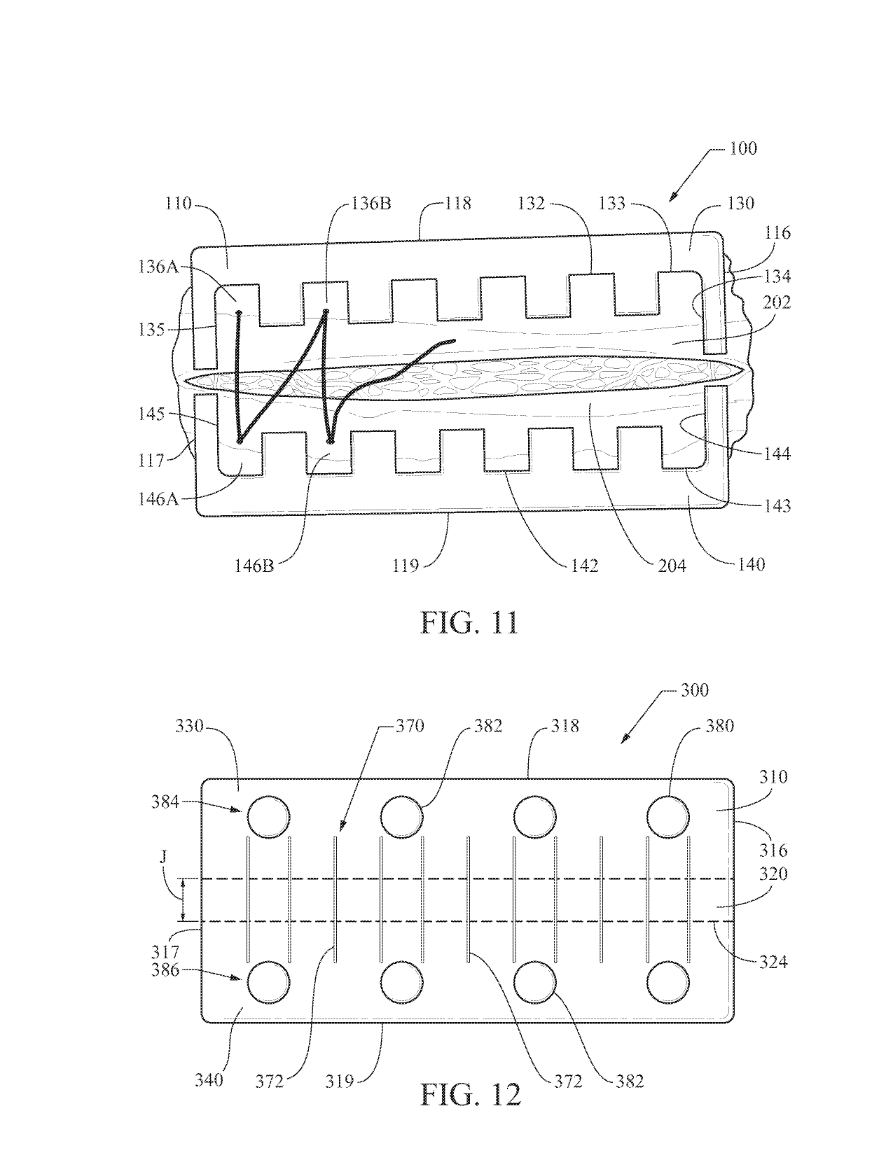

Upon completion of the incision, a first uterine tissue plane 202 and a second uterine tissue plane 204 may be disposed on opposite sides of the incision as shown in FIG. 11. The first portion 130 of the sheet member 110 may be affixed to the first uterine tissue plane 202, and the second portion 140 of the sheet member may be affixed to the second uterine tissue plane 204. After delivery of a child through the incision, the physician may approximate or align the first uterine tissue plane 202 and the second uterine tissue plane 204 with one another to close the incision through the uterine wall 200. The medical device 100 may aid in approximating the tissue planes. For example, the physician may pull the first uterine tissue plane 202 and the second uterine tissue plane 204 toward one another (e.g., with forceps). The physician may approximate the tissue planes by aligning the separated first portion 130 and second portion 140 of the sheet member 110 with one another. In one example, the physician may align the portion of an end edge (e.g., the first end edge 116 and/or the second end edge 117) of the first portion 130 of the sheet member 110 with the corresponding portion of the end edge of the second portion 140 of the sheet member 110. Additionally, or alternatively, the physician may align one or more protrusions 136 of the first portion 130 of the sheet member 110 with the corresponding protrusions 146 and/or recesses of the second portion 140 of the sheet member. By aligning the first portion 130 and the second portion 140 of the sheet member with one another, the physician may align the tissue planes attached to the respective portions of the sheet member with one another. This may aid in properly approximating the first uterine tissue plane 202 and the second uterine tissue plane 204 with one another. The tissue planes may be held together by any suitable means including, for example, forceps. In one example, the medical device may include one or more coupling members to aid in holding the tissue planes together as described below with reference to the medical device 300.

With the first uterine tissue plane 202 and the second uterine tissue plane 204 approximated, the physician may close the incision in the uterine wall 200. The physician may close the incision using any suitable technique including, for example, suturing, stapling, bonding, or any other technique. In one example, the physician may close the incision by suturing. The suture may be a single or dual layer suture. The medical device 100 may aid the physician in suturing the incision. For example, the first guide comb of the first portion 130 of the sheet member 110 and the second guide comb of the second portion 140 of the sheet member may collectively form a suture guide as shown in FIG. 11. The physician may place the sutures around the teeth of the suture guide. In one example, the physician may begin the suture, for example, in a first recess 136A of the first guide comb. The first recess 136A may be positioned between a protrusion 136 of the first portion 130 of the sheet member 110 and the second end portion 135 of the edge 132. The physician may use a needle driver to pass a needle through a portion of the uterine wall 200 aligned with the first recess 136A. The physician may pass the needle through another portion of the uterine wall 200 aligned with a first recess 146A of the second guide comb of the second portion 140 of the sheet member 110. The first recess 146A may be positioned between a protrusion 146 of the second portion 140 of the sheet member 110 and the second end portion 145 of the edge 142. In this manner, a first stitch may extend between the first recess 136A of the first portion 130 of the sheet member 110 and the first recess 146A of the second portion 140 of the sheet member as shown in FIG. 11. In other words, the first stitch may extend between a portion of the tissue plane 202 aligned with the first recess 136A of the first portion 130 and a portion of the tissue plane 204 aligned with the first recess 146A of the second portion 140.

The physician may make subsequent stitches of the suture in a similar manner. For example, the physician may pass the needle through the portion of the uterine wall 200 aligned with a second recess 136B of the first guide comb. The second recess 136B may be positioned adjacent to the recess 136A, with a protrusion 136 of the first guide comb positioned between the first recess 136A and the second recess 136B. The physician may pass the needle through the portion of the uterine wall 200 aligned with a second recess 146B of the second guide comb. The second recess 146B may be positioned adjacent to the recess 146A, with a protrusion 146 of the second guide comb positioned between the first recess 146A and the second recess 146B. In this manner, a second stitch may extend between the second recess 136B of the first portion 130 of the sheet member 110 and the second recess 146B of the second portion 140 of the sheet member as shown in FIG. 11. Subsequent stitches may be made in a similar manner such that the suture forms a zig-zag pattern along the suture guide of the medical device 100. The suture guide may function as a template for placing the suture which may aid the physician in evenly spacing the stitches. Additionally, or alternatively, the suture guide may aid in maintaining the tissue planes in alignment during suturing.

Once the incision is closed, the sheet member 110 may be removed from the uterine wall 200. In other words, the sheet member 110 may be lifted away from the outer surface of the uterine wall. The incision through the abdomen may be closed in any conventional manner. In one example, the sheet member 110 may remain in place on the uterine wall after closure of the incision. In other words, the sheet member 110 may remain implanted within the patient's body following closure of the abdomen. To that end, the medical device 100 (e.g., the sheet member, the medical adhesive, and/or the mechanical attachment mechanism) may be formed from a biodegradable material, which may dissipate upon implantation within the body. Such dissipation may occur by any method including, for example, dissolution, degradation, absorption, and excretion. The biodegradable material may include any suitable biodegradable material known in the art such as, for example, any of those described in U.S. Pat. No. 8,128,682 to Case et al., which is incorporated herein by reference. The biodegradable sheet member may degrade following closure of the abdomen. Thus, removal of the sheet member from the uterine wall prior to closure of the abdomen may be unnecessary.

FIG. 12 illustrates another example of a medical device 300. The medical device 300 may be substantially similar to the medical device 100 described above in many respects. For example, the medical device 300 may include a sheet member 310. The sheet member 310 may be a relatively thin sheet of material and may include a first surface 312 and a second surface 314 positioned opposite the first surface. The second surface 314 may be configured to engage a surface of a body tissue as described above with reference to the second surface 114. The sheet member 310 may have any size and/or shape as described above with reference to the sheet member 110.

The medical device 300 may include an incision guide 320, which may extend along the length of the sheet member 310. In one example, the incision guide 320 may extend along substantially the entire length of the sheet member 310 as shown in FIG. 12. In other examples, the incision guide may extend along any portion of the length of the sheet member 310. In one example, the incision guide 320 may include an indicator 324 as shown in FIG. 12. The indicator may include one or more marks which may be disposed on the sheet member 310 (e.g., on the first surface 312). In one example, the indicator 324 may include a pair of marks extending along the length of the sheet member 310 as shown in FIG. 12. The pair of marks may be substantially parallel to one another and spaced from one another by a width J. The spacing between the pair of marks may be sufficient for the sheet member 310 to be severed between the pair of marks as described above. In other words, the area between the pair of marks may be reserved for a scalpel to cut through the sheet member 310. In this manner, the indicator 320 may serve as an incision guide or template to guide a physician in making an incision as described above. In one example, the width J may be between about 0.5 cm and about 3 cm, preferably between about 1 cm and about 2 cm. In other examples, the pair of marks may be spaced by any other suitable width. In yet another example, the indicator 324 may be configured as a single mark extending along the length of the sheet member 310, a groove or channel (e.g. a portion having a reduced thickness) extending along the sheet member 310, or any other type of indicator configured to signal to a physician where an incision should be made.

The incision guide 320 may divide the sheet member 310 into a first portion 330 positioned on a first side of the incision guide and a second portion 340 positioned on a second side of the incision guide opposite the first side. The first portion 330 and the second portion 340 may be separated from one another by severing the sheet member 310 along the incision guide 320 as described above.

The medical device 300 may include an alignment mechanism 370, which may aid in aligning the first portion 330 and the second portion 340 of the sheet member 310 with one another after separation of the first portion and the second portion from one another. The alignment mechanism 370 may include one or more surface features disposed on the sheet member 310. With the first portion 330 and the second portion 340 of the sheet member 310 attached to one another, the surface features may extend across the incision guide 320. Severing the sheet member 310 along the incision guide 320 may sever the surface features. In other words, the portions of the surface features positioned on the first portion 330 of the sheet member 310 may be separated from the portions of the surface features positioned on the second portion 340 of the sheet member. The first portion 330 and the second portion 340 of the sheet member 310 may be realigned with one another by aligning the severed portions of the surface features.

In one example, the surface features may include one or more marks 372 disposed on the first surface 312 of the sheet member 310 as shown in FIG. 12. In one example, the marks 372 may be stamped on the sheet member 310 after it is formed (e.g., by injection molding). Each mark 372 may extend along the width of the sheet member 310 and transverse to the incision guide 320. The marks 372 may intersect the incision guide 320. In one example, the surface features may include a plurality of marks 372 that are approximately parallel to one another and spaced from one another with respect to the length of the sheet member 310. The marks 172 may resemble a set of gridlines disposed on the sheet member 310. In other examples, the marks may be disposed at any angle relative to one another or to the edges of the sheet member and at any position with respect to the length of the sheet member.

The medical device 300 may include one or more coupling members 380. The coupling members 380 may be configured to couple the first portion 330 and the second portion 340 of the sheet member 310 to one another as further described below. In one example, the coupling members 380 may include one or more magnetic members, which may be configured to magnetically couple the first portion 330 and the second portion 340 of the sheet member 310 to one another. For example, the coupling members 380 may include a plurality of magnetic buttons 382 as shown in FIG. 12. The magnetic buttons 382 may be attached to the sheet member 310. The magnetic buttons 382 may be spaced from one another about the sheet member 310. In one example, a first set 384 of magnetic buttons 382 may be positioned on the first portion 330 of the sheet member 310. The magnetic buttons 382 of the first set 384 may be spaced from one another with respect to the length of the sheet member 310. In one example, the first set of magnetic buttons 382 may be arranged in a substantially straight line positioned between the incision guide 320 and a side edge 318 of the sheet member 310 and extending along the length of the sheet member as shown in FIG. 12. Additionally, or alternatively, a second set 386 of magnetic buttons 382 may be positioned on the second portion 340 of the sheet member 310. The magnetic buttons 382 of the second set 386 may be spaced from one another with respect to the length of the sheet member 310. In one example, the second set of magnetic buttons 382 may be arranged in a substantially straight line positioned between the incision guide 320 and a side edge 319 of the sheet member 310 and extending along the length of the sheet member as shown in FIG. 12.

In one example, the second set 386 of magnetic buttons 382 may include a magnetic button corresponding to each magnetic button of the first set 384. Each magnetic button 382 of the first set 384 and the corresponding magnetic button of the second set 386 may be configured to be magnetically attracted to one another to magnetically couple the first portion 330 and the second portion 340 of the sheet member 310 to one another. To that end, the magnetic buttons 382 (or other coupling member 380) may be arranged on the first portion 330 and the second portion 340 of the sheet member 310 in substantially the same pattern. This may aid in properly aligning the first portion 330 and the second portion 340 with one another. Additionally, or alternatively, this may aid in providing a substantially uniform attractive force between the first portion 330 and the second portion 340 with respect to the length of the sheet member 310.

Each magnetic button 382 may have any suitable size and/or shape. For example, each magnetic button 382 may have a substantially cylindrical shape as shown in FIG. 12. Each magnetic button 382 may have a diameter between about 6.35 mm and about 15.88 mm, preferably between about 6.5 mm and about 15.5 mm. Additionally, or alternatively, each magnetic button 382 may have a height between about 1.6 mm and about 3.18 mm, preferably between about 1.75 mm and about 3 mm. In one example, a thickness of the sheet member 310 may be between about 1.5 and about 3 times the height of the magnetic buttons 382. This may enable the magnetic buttons 382 to be encapsulated within the sheet member 310 as further described below. In other examples, the magnetic buttons may have any other suitable shape such as, for example, a sphere, an ellipsoid, a prism (e.g., triangular, rectangular, pentagonal, or any other shaped prism), or any other geometric shape. In other examples, the magnetic buttons 382 may have any other suitable size. The size of the magnetic buttons 382 (or other coupling member 380) may depend on clinical need.

In other examples, the magnetic members may have any other suitable size and/or shape. For example, the coupling member 380 may include one or more magnetic strips. In one example, the coupling member 380 may include a plurality of magnetic strips, and one magnetic strip may extend along the length of each of the first portion 330 and the second portion 340 of the sheet member 310. In another example, the magnetic members may be configured as one or more magnetic threads which may be attached to the sheet member 310. For example, the magnetic threads may be woven into the sheet member 310 or encapsulated in the sheet member. In another example, the magnetic members may include magnetic particles. For example, the sheet member 310 may be impregnated with a plurality of magnetic particles to impart magnetic properties thereto. In another example, the sheet member 310 (or a portion thereof) may be formed from a magnetic material. In other examples, the coupling member 380 may include any number of magnetic members positioned at any suitable location with respect to the sheet member 310.

In any of the examples described herein, the magnetic members 380 (e.g., the magnetic buttons 382, the magnetic strips, or any other magnetic member) may include any type of material capable of producing a magnetic field. Additionally, or alternatively, the magnetic members may include any type of material capable of interacting with a magnetic field. In other words, the magnetic members may include a material that creates a persistent magnetic field (e.g., a magnetically hard material or a permanent magnet) or a material which does not create a persistent magnetic field, but which may be magnetized (e.g., a magnetically soft material). The magnetic members may include any type of magnetic material such as, for example, a magnetic metal or composite (e.g., nickel titanium alloy, stainless steel, iron, nickel, or cobalt), a ceramic material having magnetic properties, a polymeric material coated or impregnated with a magnetic material, or any other type of magnetic material. In one example, the magnetic members may include a rare earth magnet (e.g., a neodymium or a samarium cobalt magnet), which may be plated or coated with a metallic material such as stainless steel. In one example, the magnetic members may include an electromagnet, which may act as a magnet when an electric current is passed therethrough and cease to act as a magnet when the electric current is not passed therethrough. In this manner, the magnetic members may be magnetized and/or demagnetized (e.g., by starting or stopping the electric current) as desired during a medical procedure. The magnetic members may have any suitable magnet strength (e.g., gauss rating or pull strength). The magnet strength may depend on clinical need.

In one example, the magnetic members may be polarized to aid in proper positioning and/or alignment of the first portion 330 and the second portion 340 of the sheet member 310. For example, the magnetic members 382 of the first set 384 and/or the second set 386 may be polarized such that the attractive force between each magnetic member of one set and the corresponding magnetic member of the other set is greater than the attractive force between non-corresponding magnetic members. For example, a magnetic member 382 of the first set 384 positioned near an end edge 317 of the sheet member 310 may be more attracted to the corresponding magnetic member of the second set 386 positioned near the end edge 317 than to other magnetic members of the second set. In this manner, the magnetic members 382 may aid in biasing the first portion 330 and the second portion 340 of the sheet member 310 into alignment with one another.

The coupling members 380 (e.g., the magnetic members 382) may be attached to the sheet member 310. In one example, the coupling members may be attached to a surface (e.g., the first surface 312 or the second surface 314) of the sheet member 310. The coupling members may be attached to the surface of the sheet member by any suitable method including, for example, adhesive, sutures, staples, clamps, or any other attachment mechanism. In another example, the coupling members may be encapsulated or embedded within the sheet member 310. For example, the coupling members may be positioned within a void in the sheet member so that the sheet member at least partially surrounds the coupling members. In one example, the coupling members may be overmolded with a polymer material to form the sheet member with the coupling members encapsulated therein. In one example, the coupling members may be disposed on a substrate such as, for example, a surgical mesh. The substrate with the coupling members disposed thereon may be overmolded with a polymer material to form the sheet member with the coupling members (and the substrate) encapsulated therein. In one example, the coupling members may be entirely surrounded by the sheet member so that no portion of the coupling members is exposed on the exterior surface of the medical device. In this manner, substantially the entire exterior surface of the medical device may be covered by a relatively soft material which may reduce the risk of damaging the body tissue engaged by the medical device as described above.

The medical device 300 may be used to aid a physician in making and/or closing an incision in a body tissue during a surgical procedure. Use of the medical device 300 may be substantially similar to use of the medical device 100 as described above. For example, during a cesarean section, the medical device 300 may be placed on the outer surface of the uterine wall. The second surface 314 of the sheet member 310 may engage the outer surface of the uterine wall as described above with reference to the second surface 114.

With the sheet member 310 in place on the outer surface of the uterine wall, the physician may make an incision through the uterine wall. For example, the physician may cut into the sheet member 310 and the uterine wall using the incision guide 320 to guide the incision as described above. The physician may cut the sheet member 310 and the portion of the uterine wall aligned with the sheet member along the incision guide 320 from the first end edge 316 of the sheet member and along the incision guide 320 to the second end edge 317 of the sheet member as described above to separate the first portion 330 and the second portion 340 of the sheet member from one another and to complete the incision in the uterine wall.

Upon completion of the incision, the first portion 330 of the sheet member 310 may be affixed to the first uterine tissue plane, and the second portion 340 of the sheet member may be affixed to the second uterine tissue plane as described above. After delivery of a child through the incision, the physician may approximate or align the first uterine tissue plane and the second uterine tissue plane with one another to close the incision through the uterine wall. The medical device 100 may aid in approximating the tissue planes. For example, the physician may pull the first and second uterine tissue planes toward one another and approximate the tissue planes by aligning the separated first portion 330 and second portion 340 of the sheet member 310 with one another. In one example, the physician may use the alignment mechanism 370 to align the first portion 330 and the second portion 340. For example, the physician may align the portion of the severed mark 372 positioned on the first portion 330 of the sheet member 210 with the corresponding portion of the severed mark positioned on the second portion 340 of the sheet member to align the first and second portions with one another. By aligning the first portion 330 and the second portion 340 of the sheet member 310 with one another, the physician may align the tissue planes attached to the respective portions of the sheet member with one another. This may aid in properly approximating the uterine tissue planes with one another.

In one example the first portion 330 and the second portion 340 of the sheet member 310 may be coupled to one another upon alignment of the tissue planes as described above. For example, the coupling member 380 of the first portion 330 may be coupled to the corresponding coupling member of the second portion 340. In one example, the first portion 330 and the second portion 340 of the sheet member 310 may be magnetically coupled to one another. The uterine tissue planes may be held together by the coupled first portion 330 and second portion 340 of the sheet member 310. In other words, the first portion 330 and the second portion 340 may be coupled to one another to hold the incision closed while maintaining substantial alignment of the tissue planes on opposite sides of the incision. With the uterine tissue planes approximated, the physician may close the incision in the uterine wall as described above (e.g., by suturing). Once the incision is closed, the sheet member 310 may be removed from the uterine wall or left in place on the uterine wall as described above. The incision through the abdomen may be closed in any conventional manner.

In any of the examples described herein, the sheet member (e.g., the sheet member 110 or the sheet member 310) may be formed from any suitable material. Preferably, the sheet member may be formed from a material that is relatively easy to cut with a scalpel as described above. To that end, the sheet member may be formed from a soft material that is relatively easy to shear. In one example, the incision guide may be formed from a material that is different from the remainder of the sheet member, and the material of the incision guide may be more easily cut than the material of the remainder of the sheet member.

In one example, the sheet member may be formed from a polymeric material such as, for example, silicone, vinyl, latex, acrylonitrile butadiene styrene (ABS), polyurethane, polyether ether ketone (PEEK), polyethylene terephthalate (PET), polyethylene oxide (PEO), polystyrene, polyamide, polyvinyl chloride (PVC), polytetrafluoroethylene (PTFE), expanded polytetrafluoroethylene (ePTFE), polypropylene, high density polyethylene (HDPE), an elastomer (e.g., a viscoelastic polymer), or any other suitable polymeric material.

In another example, the sheet member may be formed from a remodelable material. Suitable remodelable materials may be provided by collagenous extracellular matrix (ECM) materials possessing biotropic properties. For example, suitable collagenous materials may include ECM materials such as those including submucosa, renal capsule membrane, dermal collagen, dura mater, pericardium, fascia lata, serosa, peritoneum or basement membrane layers, including liver basement membrane. Suitable submucosa materials for these purposes may include, for example, intestinal submucosa including small intestinal submucosa (e.g., Surgisis® Biodesign™, Cook Medical Incorporated, Bloomington, Ind.), stomach submucosa, urinary bladder submucosa, and uterine submucosa. For additional information as to some of the materials which may be useful in the present invention, and their isolation and treatment, reference can be made, for example, to U.S. Pat. Nos. 4,902,508, 5,554,389, 5,993,844, 6,206,931, and 6,099,567.

In another example, the sheet member may be formed from a metallic material such as, for example, a cobalt-chromium alloy (e.g., cobalt-chrome), nitinol, stainless steel, or any other suitable metallic material. In other examples, the sheet member may be formed from any other suitable material such as, for example, pyrolytic carbon. Preferably, the sheet member may be formed from a material having a relatively high tear strength. Additionally, or alternatively, the sheet member may be formed from a material having a low modulus of elasticity.

The sheet member may be formed using any suitable process. In one example, the sheet member may be formed using a molding process (e.g., injection molding). In another example, the sheet member may be configured as a laminate material. In other words, the sheet member may include a plurality of layers or sheets of material cooperatively forming the sheet member. In another example, the sheet member may be formed as a weave or mesh of filamentary strands or wires (e.g., polymeric or metallic strands or wires).

While the medical devices have been described herein with reference to approximating uterine tissue during a cesarean section, this disclosure is not so limited. The medical devices described herein may be used to approximate planes of any body tissue to aid in closure of an incision therethrough. For example, the medical device may be used to aid in closure of an opening formed in any internal organ with a cavity such as, for example, the stomach, the digestive tract, or the urinary tract.

While various embodiments of the invention have been described, the invention is not to be restricted except in light of the attached claims and their equivalents. Moreover, the advantages described herein are not necessarily the only advantages of the invention and it is not necessarily expected that every embodiment of the invention will achieve all of the advantages described.

We claim:

1. A medical device comprising:
a sheet member comprising a first surface and a second surface opposite the first surface to engage a surface of a body tissue;
an incision guide extending along a length of the sheet member, a first portion of the sheet member positioned on a first side of the incision guide and a second portion of the sheet member positioned on a second side of the incision guide opposite the first side, the sheet member being severable along the incision guide to separate the first portion of the sheet member and the second portion of the sheet member from one another;
a first magnetic member positioned on the first portion of the sheet member, and a second magnetic member positioned on the second portion of the sheet member, the first magnetic member and the second magnetic member being attracted to one another to magnetically couple the first portion of the sheet member and the second portion of the sheet member to one another; and
an alignment mechanism for aligning the first portion of the sheet member with the second portion of the sheet member after separation of the first portion and the second portion from one another.

2. The medical device of claim 1, wherein the incision guide comprises a mark disposed on the first surface of the sheet member and extending along the length of the sheet member.

3. The medical device of claim 1, wherein the incision guide comprises a groove in the first surface of the sheet member and extending along the length of the sheet member.

4. The medical device of claim 1, wherein the alignment mechanism comprises a surface feature disposed on the first surface of the sheet member and extending along a width of the sheet member, and the surface feature intersects the incision guide.

5. The medical device of claim 4, wherein the surface feature comprises a plurality of surface features spaced from one another with respect to the length of the sheet member, and each surface feature intersects the incision guide.

6. The medical device of claim 1, wherein the second surface comprises a medical adhesive to engage the surface of the body tissue.

7. The medical device of claim 1, wherein the first magnetic member comprises a first set of magnets spaced from one another with respect to the length of the sheet member.

8. The medical device of claim 7, wherein the second magnetic member comprises a second set of magnets spaced from one another with respect to the length of the sheet member.

9. The medical device of claim 8, wherein, with the first portion and the second portion of the sheet member magnetically coupled to one another, each magnet of the first set is substantially aligned with a corresponding magnet of the second set.

10. The medical device of claim 1, wherein each of the first magnetic member and the second magnetic member is encapsulated within the sheet member.

11. A medical device comprising:
a sheet member comprising a first surface and a second surface opposite the first surface to engage a surface of a body tissue; and
an incision guide extending along a length of the sheet member, a first portion of the sheet member positioned on a first side of the incision guide, a second portion of the sheet member positioned on a second side of the incision guide opposite the first side, the incision guide comprising an opening through the sheet member extending along a portion of a length of the incision guide;
wherein the first portion of the sheet member comprises a first edge extending along the opening, the second portion of the sheet member comprises a second edge extending along the opening and positioned opposite the first edge, the first edge comprises a first projection, and the second edge comprises a second projection; and
wherein the sheet member is severable along the incision guide to separate the first portion and the second portion of the sheet member from one another, and the first projection and the second projection are positionable relative to one another to realign the first portion and the second portion of the sheet member with one another after separation of the first portion and the second portion from one another.

12. The medical device of claim 11, wherein the incision guide comprises a first indicator and a second indicator, each of the first indicator and the second indicator is disposed on the first surface of the sheet member and extends along the length of the sheet member, and the opening is positioned between the first indicator and the second indicator with respect to the length of the sheet member.

13. The medical device of claim 11, wherein the first projection extends into the opening in a first direction, and the second projection extends into the opening in a second direction opposite the first direction.

14. The medical device of claim 11, wherein the first projection and the second projection are arranged relative to one another such that the first projection is substantially aligned with the second projection.

15. The medical device of claim 11, wherein the first projection and the second projection are arranged relative to one another such that the first projection is offset from the second projection.

16. The medical device of claim 11, wherein the first projection and the second projection collectively define a suture guide for guiding placement of sutures for closing an incision aligned with the opening of the incision guide.

17. The medical device of claim 11, wherein the first projection comprises a first plurality of projections, the second projection comprises a second plurality of projections, and each of the first plurality of projections is positioned opposite a corresponding one of the second plurality of projections with respect to the opening of the incision guide.

18. The medical device of claim 17, wherein each projection of the first plurality of projections is separated from an adjacent projection of the first plurality of projections by a distance of between about 5 mm and about 8 mm, and each projection of the second plurality of projections is separated from an adjacent projection of the second plurality of projections by a distance of between about 5 mm and about 8 mm.

19. The medical device of claim 17, wherein the first plurality of projections comprises a first guide comb extending along the opening of the incision guide, and the second plurality of projections comprises a second guide comb extending along the opening of the incision guide.

20. A method for performing a surgical procedure, the method comprising:
applying a medical device to a surface of a body tissue, the medical device comprising a sheet member comprising an engaging surface engaged with the surface of the body tissue;
cutting along an incision guide of the medical device to make an incision in a portion of the body tissue aligned with the incision guide, a first plane of the body tissue positioned on a first side of the incision, and a second plane of the body tissue positioned on a second side of the incision opposite the first side;
separating a first portion of the sheet member positioned on a first side of the incision guide and engaged with the first plane of the body tissue from a second portion of the sheet member positioned on a second side of the incision guide opposite the first side and engaged with the second plane of the body tissue to separate the first plane of the body tissue and the second plane of the body tissue from one another;
magnetically coupling the first portion of the sheet member and the second portion of the sheet member to one another to align the first plane of the body tissue and the second plane of the body tissue with one another.
